(19) United States
(12) Patent Application Publication
(10) Pub. No.: US 2006/0062854 A1
(43) Pub. Date: Mar. 23, 2006

(54) Hydrogel having anti-microbial properties

(75) Inventors: N. Satish Chandra, Lansdale, PA (US); William F. McNally, Clarks Summit, PA (US); Joel M. Furey, Stowe, VT (US); Gregory J. Robb, Lawrenceville, NJ (US); Matthew L. Harriton, New York, NY (US)

Correspondence Address: AKERMAN SENTERFITT, P.O. BOX 3188, WEST PALM BEACH, FL, 33402-3188, US

(73) Assignee: Noble Fiber Technologies, LLC, Scranton, PA (US)

(21) Appl. No.: 11/233,339

(22) Filed: Sep. 22, 2005

Related U.S. Application Data
(60) Provisional application No. 60/611,959, filed on Sep. 22, 2004.

(51) Int. Cl. A61K 9/14; A61K 33/38

(52) U.S. Cl. 424/486; 424/488; 424/618

(57) ABSTRACT

A method of making a hydrogel having antimicrobial properties. The hydrogel includes a hydrogel-forming polymer and an anti-microbial agent. The method includes mixing a hydrogel-forming polymer, such as a hydrophilic polymer, with water and cross-linking the polymer and water using an energy source. The method does not require any chemical additive to affect the cross-linking. The anti-microbial agent may be mixed with the hydrogel-forming polymer and water prior to cross-linking. Alternatively, the anti-microbial agent may be applied to a substrate onto which the hydrogel is placed such that the anti-microbial agent or the properties of the agent, or both, migrate into the hydrogel. The substrate may be a liner onto which the hydrogel is placed, a scrim located on or in the hydrogel, or other appropriate device.

### CROSS-REFERENCE TO RELATED APPLICATIONS

This application claims the benefit of U.S. Provisional Patent Application No. 60/611,959, filed Sep. 22, 2004.

### FIELD OF THE INVENTION

This invention is directed generally to hydrogels, and more particularly to methods of making hydrogels having anti-microbial properties.

### BACKGROUND OF THE INVENTION

A wide variety of disposable absorbent articles designed not only to be efficient in the absorption of body fluids such as urine, blood, menses and the like, but also to be sanitary and comfortable in-use, are known in the literature. Disposable absorbent products of this type generally comprise a fluid-permeable topsheet material, an absorbent core, and a fluid-impermeable backsheet material. Various shapes, sizes and thicknesses of such articles have been explored in an attempt to make their use more comfortable and convenient.

More recently, research has been focused on the removal of foul odors and the prevention of skin diseases such as dermatitis, rash and redness caused by wearing a disposable absorbent article for a relatively long time. Many body fluids have an unpleasant odor, or develop such odors when in contact with air and/or bacteria for prolonged periods. Additionally, urine and/or other exudates absorbed into the absorbent article are converted to ammonia by urease produced by skin-flora, i.e., a group of normal microorganisms on the skin. This ammonia, in turn, causes dermatitis, rash and/or other forms of skin irritation. Such disease of the skin in infants can be a serious medical matter which, in extreme cases, can result in death.

Additionally, superficial topical infections are typically a consequence of a primary disease source such as chronic urinary incontinence, or are directly related to a contagious nosocomial or endemic source. Prolonged moist or wet skin conditions often lead to maceration and other changes in skin integrity which provide the opportunity for normally saprophytic bacteria and fungi to invade the site and establish an infection.

Wounds that are heavily contaminated by microorganisms, but not clinically infected, are often characterized by a prolonged period of inflammation, as well as a delay in wound repair and healing. Microorganisms that contaminate wounds have been implicated as an important factor in the retardation of wound healing by interfering with leucocyte phagocytosis, and by the depletion of nutrients and oxygen required for normal tissue granulation.

A large number of dressings, bandages, and topic medicaments are available for the treatment of these and other types of wounds. These products fall into two categories, passive and active. Passive wound dressings are dressing which serve only to provide mechanical protection and a barrier to infection. The dressings themselves do not supply any composition which enables or facilitates the healing process of the wound. Examples of passive dressings include gauze and adhesive bandages. Active dressings are dressings that supply some biologically active compound to the site of a wound. One type of active dressing is a dressing or wrapping that delivers or has been impregnated with antimicrobials (e.g., Bacitracin).

Another family of dressings that contain both passive and active properties is the hydrogels or hydrocolloids. Although many of these dressings do not supply any biologically active compound to the wound, they are specifically designed to create a moist environment around the wound to promote wound healing. Hydrogel and hydrocolloid dressings have been formulated to antimicrobials to help prevent and/or treat infection. However, to date, hydrogels or hydrocolloids have been difficult to form as either a chemical additive has been needed to chemically cross-link the hydrogel materials or to provide an enhancer for cross-linking using an source of energy, such as ultraviolet light. These chemical additives increase the cost and/or the complexity of using hydrogels.

It has been suggested that the topical application of biological compounds may play an active role in wound healing. These compounds include mitogens, cytokines, growth factors, and hormones. However, there are limitations to these therapies. First, it is difficult to regulate the dosage of such an application. A liquid or viscous paste containing these components applied to a wound will tend to spread away from the site of the wound, or will be absorbed by and removed from the wound by dressings which are placed over the wound. Dressings that come in contact with the wound surface may also interfere with the normal healing process. Furthermore, since these compounds are all polypeptides, they are extremely susceptible to rapid degradation following there application. Such degradation can occur from the contact of the polypeptides with proteases produced by bacteria normally on the surface of the skin. In addition, these agents may lack specificity in there action, and have adverse pleiotropic effects on adjacent tissues other than those tissues involved in wound healing. These problems may be alleviated through the use of hydrogels but, as previously discussed, there are issues of cost and complexity involved in the formation of hydrogels using today's processes.

Accordingly, what is needed is a material that has antimicrobial activity without the problems associated with prior art solutions. Also what is needed is a material having antimicrobial activity that may be used in a variety of different applications. Also what is needed is a method of making hydrogel materials that is easier and/or more efficient than prior art methods.

### SUMMARY OF THE INVENTION

The present invention provides a method of making a hydrogel having antimicrobial activity. The hydrogel includes a hydrogel-forming polymer and an anti-microbial agent comprising a silver-coated fiber. The method mixes a hydrogel-forming polymer, such as a hydrophilic polymer, with water and cross-links the polymer and water using an energy source. The method does not need any chemical additive to affect the cross-linking, unlike prior art methods of forming hydrogels. The anti-microbial agent may be mixed with the hydrogel-forming polymer and water prior to cross-linking. Alternatively, the anti-microbial agent may be applied to a substrate onto which the hydrogel is placed such that the anti-microbial agent migrates into the hydrogel.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a perspective view of a substrate with a hydrogel layer positioned on top of the substrate.

FIG. 2 is an exploded perspective view of an alternative configuration of a substrate and hydrogel layers.

### DETAILED DESCRIPTION OF THE INVENTION

The present invention is directed to a method of making a hydrogel that has anti-microbial properties. The method includes mixing a hydrogel-forming polymer, such as a hydrophilic polymer, with water and cross-linking the polymer and water using an energy source. The method does not require use of any chemical additive to affect the cross-linking. As such, the methods of the present invention are more efficient and/or cost-effective because there is no need for a chemical additive to enhance cross-linking as with prior art methods. In addition, the hydrogel materials may include an anti-microbial agent.

In a first aspect, the present invention provides a method of making a hydrogel wherein a hydrogel-forming polymer is mixed with water. In one embodiment, the hydrogel-forming polymer is a hydrophilic polymer. The hydrogel-forming polymer may be mixed with water in a wide range of ratios. The hydrogel-forming polymer may be mixed with water in a ratio range of from about one part hydrogel-forming polymer to about thirty-three parts of water, by weight to about one part hydrogel-forming polymer to about three parts of water.

In one embodiment, the hydrogel-forming polymer is a hydrophilic polymer. Examples of hydrophilic polymers that may be used include, but are not limited to, starch, cellulose, cellulose derivatives, polyvinyl alcohol, polyalkylene oxide, polyethylene oxide, polypropylene glycol, and other hydrophilic polymers including, but not limited to, poly(1,3-dioxolane), copolymers of polyethylene oxide or poly(1,3-dioxolane), polyvinyl pyrrolidone, polyethylene glycol, polyacrylic acid poly(2-methyl-2-oxazoline polyglycidyl trimethyl ammonium chloride, polymethylene oxide, and the like. In one embodiment, the hydrophilic polymer may be a polyethylene oxide.

Once the hydrogel-forming polymer and water are mixed, the mixture is then cross-linked to cause the hydrogel-forming polymer to cross-link with the water to form the hydrogel of the present invention. The cross-linking is accomplished using an energy source. The mixture is subjected to this energy source. Depending on the hydrogel-forming polymer used, the ratio of the hydrogel-forming polymer and water, and the amount of anti-microbial agent and/or additional additives, the amount of energy needed to cause cross-linking may vary. In general, the amount of energy used in the present invention is any amount sufficient to cause the hydrogel-forming polymer and water to form cross-linked bonds to give the hydrogel greater molecular integrity and/or thereby creating a stable sheet of hydrogel.

The energy source used in the present invention may be any energy source capable of causing the cross-linking to occur. In one embodiment, the energy source is an electron beam, such as one generated by an electron beam accelerator. In another embodiment, the energy source is gamma radiation. Other energy sources that can cause cross-linking bonds between the hydrogel-forming polymer and the water may also be used in the present invention.

The energy may be supplied in an amount sufficient to cause cross-linking. The exact amount of energy used in forming the hydrogel is dependent on process considerations and a variety of different factors. In one embodiment, the hydrogel is formed by placing the mixture on a substrate and supplying the energy source as the substrate is passed through the energy source. The process parameters may vary depending on the line speed, the strength of the energy field, or the amount of energy needed to cross-link the hydrogel-forming polymer and water, or any combination of these items. For example, in one embodiment, the strength of the energy field may be about 40 mA, and the line speed may be varied as needed depending on the amount of energy needed to cross-link the hydrogel. However, in another embodiment wherein the energy field is as low as 0.1 mA, the line speed may be much slower, especially if a larger amount of energy is needed to cause cross-linking of the hydrogel-forming polymer with the water.

The present invention may also include an anti-microbial agent. In one embodiment, the anti-microbial agent may be silver. The silver may be in pure form, or may be included with another material in another form, such as a silver fiber or silver powder. The silver fiber may be in fiber or mesh form. The silver may be produced by depositing silver by various methods on a flexible substrate such as nylon, polyester, and the like. Methods for depositing silver onto a surface may include chemical deposition, electroplating, or vacuum deposition.

The hydrogel-forming polymer, water and the anti-microbial agent may include one or more additives that are added to the hydrogel-forming polymer/water mixture to assist in making the hydrogels or to affect the characteristics of the final material based upon the expected end-use for the product, or both. For example, if the hydrogel is used with an article that contacts the skin, a pH adjuster may be added to reduce or increase irritation of the skin. Other additives may include an anti-fungal additive or another anti-microbial additive for further anti-microbial protection; preservatives; or a salt to increase the conductivity of the hydrogel, or any combination thereof. Alternatively, in some embodiments, it may be beneficial to decrease the rate of cross-linking. As such, a cross-linking inhibitor may be used. In other embodiments, it may be beneficial to add a cross-linking enhancer. The total amounts of these additives, whether it be one additive or a combination of additives, may range from about 0 to about 10 percent by weight of the hydrogel. In alternative embodiments, these additives may range from about 0.5 percent to about 5 percent by weight of the hydrogel.

While the methods of the present invention may be performed without an enhancer, there may be instances wherein small amounts, i.e. less than about one percent by weight, of a cross-linking enhancer may be used. The resulting hydrogel is substantially free from any cross-linking enhancer additive. Accordingly, as used herein, the term “substantially free of any additive for enhancing cross-linking” means a hydrogel having less than about one percent by weight of the hydrogel.

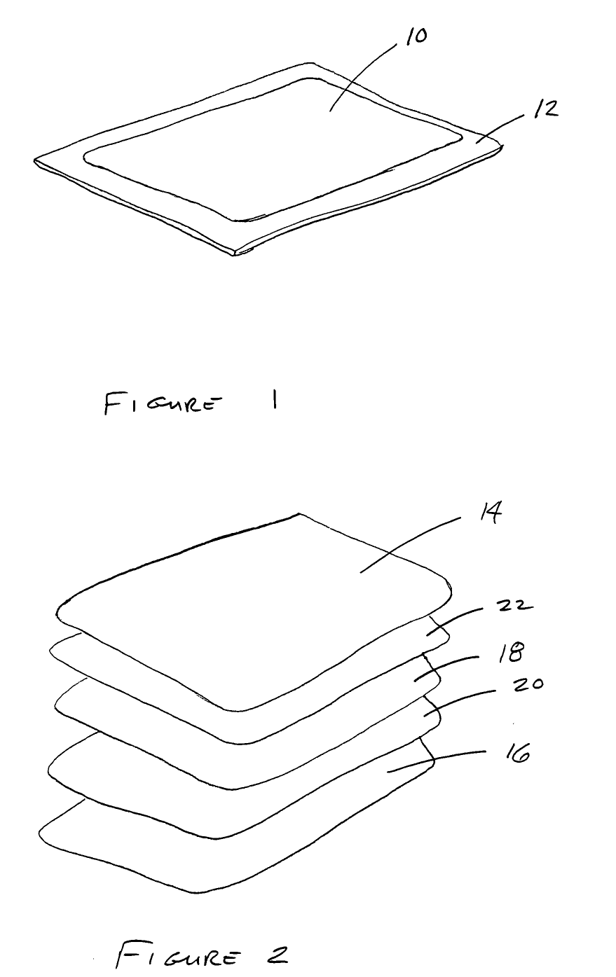

As shown in FIG. 1, the hydrogel 10 may be applied to any substrate 12. The hydrogel mixture 10 may be placed onto a substrate 12 prior to cross-linking or after cross-linking. The substrate 12 may be a top liner 14, a bottom liner 16, a scrim 18, or a combination thereof. The hydrogel 10 may also be placed such that there are multiple layers of hydrogel 10 with a substrate layer 12 between the hydrogel layers. For example, in one embodiment, as shown in FIG. 2, the hydrogel 10 may be placed on a bottom layer 20, with a scrim 18 placed on the layer of hydrogel 10. Then, another layer of hydrogel 22 may be applied, followed by a top liner 14. In this embodiment, the scrim 18 is embedded within the two hydrogel layers 20, 22. The top liner 14 or the bottom liner 16 may be made from any suitable material, such as a polyester film, a polyethylene film, or the like. In certain embodiments, the anti-microbial agent is applied to the substrate 12 rather than being mixed with, or in addition to mixing with, the hydrogel-forming polymer and water. As a result, the anti-microbial agent or properties of the agent, or both, may migrate into the hydrogel 10, thereby providing the anti-microbial properties to the hydrogel 10. If multiple substrates are used in a particular embodiment, one or more of the substrates 12 may have the anti-microbial agent applied thereto.

While the hydrogel 10 may be applied to a substrate 12 including a top sheet liner 14 or a bottom sheet liner 16, or both, the hydrogel 10 may include a scrim 18 for supporting the hydrogel 10. The scrim 18 may be any material capable of providing support whether the scrim is located on an external surface of the hydrogel 10 or embedded within the hydrogel 10. The scrim 18 is selected such that it may be cut, sized or otherwise manipulated such that the hydrogel 10 may include the scrim 18 when used in the final end application. The scrim 18 may be a mesh, a foam, a film, a woven material, and/or a non-woven material. In one embodiment, the scrim 18 is a high-density expanded polyethylene web.

The hydrogel 10 may be used in any application wherein it is advantageous to have anti-microbial properties. For example, the hydrogel 10 may be used in cosmetics, disinfectants, sanitizers, hospital and medical uses, bandages and wound dressings, disposable diapers, and feminine care articles.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A method of making a hydrogel having anti-microbial properties comprising:
mixing a hydrogel-forming polymer with water; and
applying an energy source to crosslink the hydrogel-forming polymer with the water to form the hydrogel;
wherein the hydrogel further includes an anti-microbial agent; and
wherein the hydrogel is substantially free of any additive for enhancing cross-linking.

2. The method of claim 1, wherein the hydrogel-forming polymer is a hydrophilic polymer.

3. The method of claim 2, wherein the hydrophilic polymer is selected from the group consisting of starch, cellulose, cellulose derivatives, polyvinyl alcohol, polyalkylene oxide, polyethylene oxide, polypropylene glycol, poly(1,3-dioxolane), copolymers of polyethylene oxide, copolymers of poly(1,3-dioxolane), polyvinyl pyrrolidone, polyethylene glycol, polyacrylic acid, and polymethylene oxide.

4. The method of claim 1, wherein the hydrogel-forming polymer is added to the water in a weight ratio ranging from about one part hydrogel-forming polymer to about thirty-three parts water to about one part hydrogel-forming polymer to about three parts water.

5. The method of claim 1, wherein the anti-microbial agent comprises silver.

6. The method of claim 5, wherein the anti-microbial agent comprises silver coated fibers.

7. The method of claim 1, wherein the anti-microbial agent is added to the hydrogel in an amount of from about 0.1 percent to about 10 percent by weight.

8. The method of claim 7, wherein the anti-microbial agent is added to the hydrogel in an amount of from about 0.1 percent to about 3 percent by weight.

9. The method of claim 1, wherein the anti-microbial agent is mixed with the hydrogel-forming polymer and the water before application of the energy source.

10. The method of claim 1, wherein the mixture of the hydrogel-forming polymer and the water is applied to a substrate before application of the energy source.

11. The method of claim 10, wherein the substrate is selected from a group consisting of a top sheet, a bottom sheet, and a scrim.

12. The method of claim 11, wherein the substrate comprises a scrim selected from a group consisting of a mesh, a foam, a film, a woven material, and a non-woven material.

13. The method of claim 12, wherein the scrim comprises a high-density expanded polyethylene web.

14. The method of claim 1, further comprising an additive selected from a group consisting of an anti-fungal additive, an anti-microbial additive, a salt, a preservative, a pH adjuster, and a cross-linking inhibitor.

15. The method of claim 1, wherein the energy source is selected from a group consisting of an electron beam and gamma radiation.

16. The method of claim 15, wherein the energy source comprises a linear accelerator electron beam.

17. A hydrogel having anti-microbial properties made according to the process of claim 1.

18. A personal care product having anti-microbial properties, comprising:
a hydrogel formed by mixing a hydrogel-forming polymer with water and applying an energy source to crosslink the hydrogel-forming polymer with the water to form the hydrogel, wherein the hydrogel further includes an anti-microbial agent and wherein the hydrogel is substantially free of any additive for enhancing cross-linking.

19. The personal care product of claim 18, wherein the personal care product is selected from a group consisting of a bandage, a cosmetic, a topical skin treatment, a wound dressing, a diaper, and a feminine care article.
